(19) United States
(12) Patent Application Publication
(10) Pub. No.: US 2012/0283801 A1
(43) Pub. Date: Nov. 8, 2012

(54) APPARATUS FOR DETECTING THE PRESENCE OF SKIN

(75) Inventors: Simon James Fisher, Pennllergaer (GB); Jan Simonsen, Struer (DK); Hanne Simonsen, Struer (DK)

(73) Assignee: CYDEN LTD., Swansea, WA (GB)

(21) Appl. No.: 12/596,704

(22) PCT Filed: Apr. 21, 2008
(86) PCT No.: PCT/GB08/50278

§ 371 Date: Sep. 15, 2011

(30) Foreign Application Priority Data
Apr. 20, 2007 (DK) 200700581

(51) Int. Cl. A61N 5/06

(52) U.S. Cl. 607/88

(57) ABSTRACT

The apparatus comprises
a) a set of probes each having tips arranged to simultaneously touch the skin and define a predetermined pattern on the skin, at least one of the probes being arranged to transmit a pulsed electrical signal and at least one of the probes (which me be the same as or different to the transmitter probe) being arranged to receive the transmitted electrical signal;
b) a signal detector for detecting the or each received electrical signal;
c) means for comparing a numerical value obtained from at least one detected signal from the signal detector with at least one predetermined numerical value; and
d) means for providing an output when said value obtained from the detected signal differs from the predetermined numerical value by more than a predetermined amount.

The apparatus is used to control intense pulsed light devices used for local treatment of various skin conditions and to influence non-desired hair growth.

The present invention relates to apparatus for detecting the presence of skin, typically for use as a safety device to ensure that light therapy devices are not used where there is no target area of skin present. The invention also encompasses a method for detecting of the presence of skin suitable for use in controlling operation of such light therapy devices, both for therapeutic purposes and for cosmetic purposes).

Various types of light therapy are known. For example, intense-pulsed-light (IPL) is used for local treatment of various skin conditions and to influence non-desired hair growth.

It is important to ensure that the light is directed at the area of skin to be treated, and not for example, at more delicate body parts (such as the eyes) and not used where skin is not detected.

US2004/0167502 A1 discloses a device and method for sensing the presence of human skin by evaluating the spectral remittance of electromagnetic radiation from the surface in question. Such a device requires complicated circuitry and optical control, separate from the optical control needed in apparatus for dermatological treatment. It is an object of the invention to provide simplified apparatus for this purpose.

According to a first aspect of the invention, there is provided apparatus for detecting the presence of skin, which apparatus comprises

a) a set of probes each having tips arranged to simultaneously touch the skin and define a predetermined pattern on the skin, at least one of said probes (hereinafter referred to as a transmitter probe) being arranged to transmit a pulsed electrical signal and at least one of said probes (hereinafter referred to as a receiver probe) being arranged to receive said transmitted electrical signal;
b) signal detector means for detecting the or each received electrical signal;
c) means for comparing a numerical value obtained from at least one detected signal from said signal detector means with at least one predetermined numerical value; and
d) means for providing an output when said value obtained from the detected signal differs from said predetermined numerical value by more than a predetermined amount.

The receiver probe may be the same as the transmitter probe, or it may be different thereto. If the receiver probe and transmitter probe are the same, the apparatus may take measurements based on capacitance (such as elapsed time to reach a predetermined voltage). In this embodiment, it is preferred that all of the set of probes are both transmitter probes and receiver probes.

If the transmitter probe and receiver probe are different probes, it is preferred to provide a single transmitter probe, with the remainder of the probes being receiver probes.

In an advantageous embodiment of the invention, the tips of the probes are fixed to a support, such that the tips together define a two dimensional pattern, such as a rectangle, in which all the tips are intended to be in contact with the skin. This pattern preferably defines the perimeter of an area that is slightly larger than the area of an aperture of an intense-pulsed-light device, such as a discharge lamp, which is to be contacted with the skin. Such a pattern is typically a rectangle measuring about 10 to 15 mm×20 to 30 mm. An arrangement of the nature just described ensures that the aperture is not obscured by the probes.

The probes themselves are preferably elongate pins, typically having a diameter of about 2 to 3 mm.

Because dry skin, oily skin and gel-covered skin all have different electrical properties, including conductance and capacitance, it is preferred that the predetermined amount is sufficiently distinct from the predetermined value in order to provide a margin of error. For example, if the predetermined value is voltage, it is preferred that the minimum of the range be above the minimum known for skin. If the predetermined value relates to capacitance, the predetermined value is preferably based on elapsed time to reach a threshold voltage which, in turn will depend on the capacitance of the skin contacted by the probe.

The apparatus according to the invention may be used for the detection of the presence of skin in an automated decision stage of a microprocessor to activate an intense-pulsed-light unit when pre-determined input conditions are fulfilled (that is when skin having parameters within a predetermined range is detected).

An advantageous use of the apparatus is to automatically determine whether the parameters measured indicate that a gel-like substance has been applied to the skin. In this case, the firing of the intense-pulsed-light is only permitted when such gel-coated skin is detected.

The output provided when the values are outside a predetermined range may comprise an alarm, which can typically be one or more of an audio signal, a visible signal or a movement signal (such as a vibration or other movement of the apparatus). Alternatively, the output may be a feedback signal arranged to cause an intense-pulsed-light source to cut out when values outside the predetermined range are detected.

According to a further aspect of the invention, there is provided a method for controlling the operation of an intense-pulsed-light source, which method comprises applying a set of probes each having tips simultaneously touching a test surface so as to define a predetermined pattern on the test surface,

transmitting a pulsed electrical signal from at least one of the probes;
receiving the signal by at least one of the probes (which may be the same as, or different to, the probe which transmits the signal),
providing connection of each of the probes receiving the transmitted pulsed electrical signal to a respective individual signal detector,
comparing a value of the detected signals from the or each signal detector with at least one predetermined value; and
providing an output when the compared value is outside a predetermined range, which output either prevents or permits the operation of the light source.

Preferred embodiments of the present invention will be further described, by way of example only, with reference to the accompanying drawings, in which

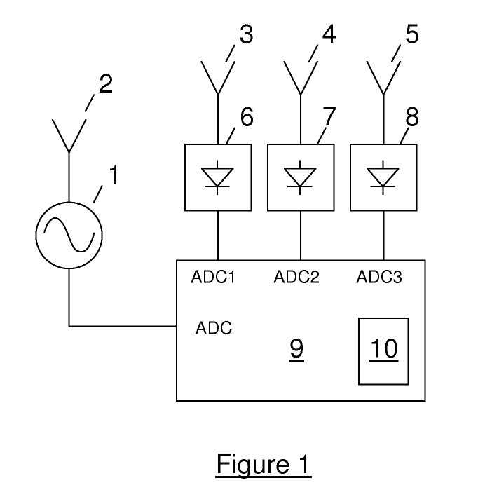

FIG. 1 is a schematic drawing of apparatus according to a first embodiment of the invention;

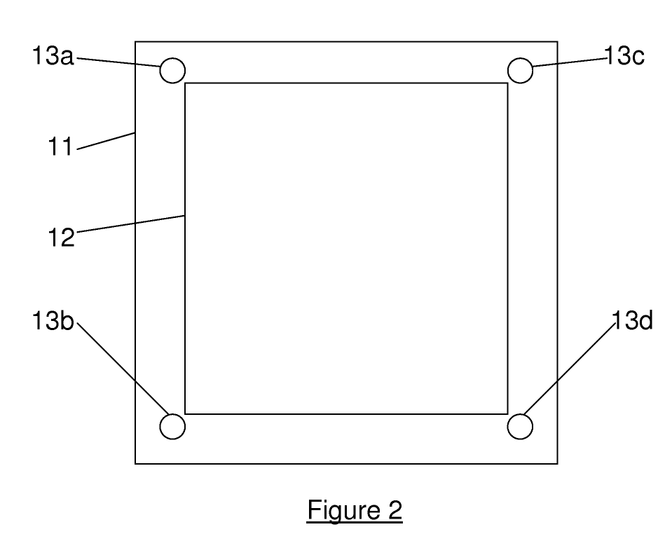

FIG. 2 is a schematic representation of four probes used in apparatus according to the invention;

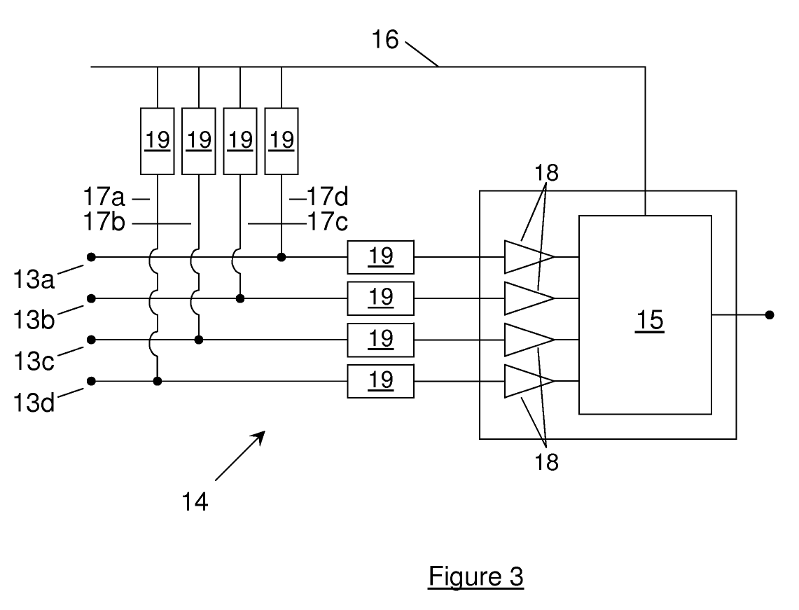

FIG. 3 is a circuit diagram of apparatus according to a second embodiment of the invention;

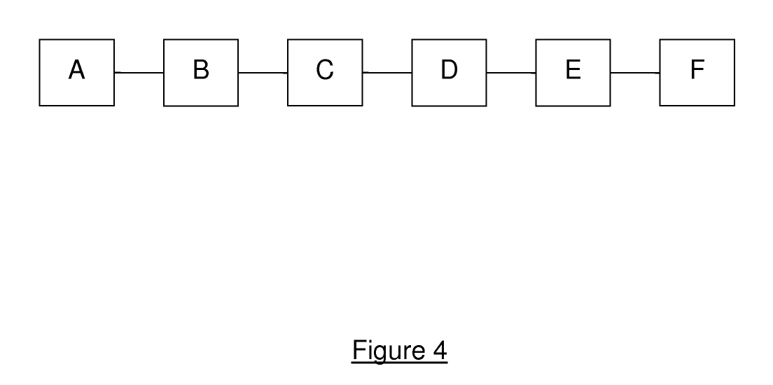

FIG. 4 is a block diagram showing preferred aspects of operation of apparatus according to the invention; and,

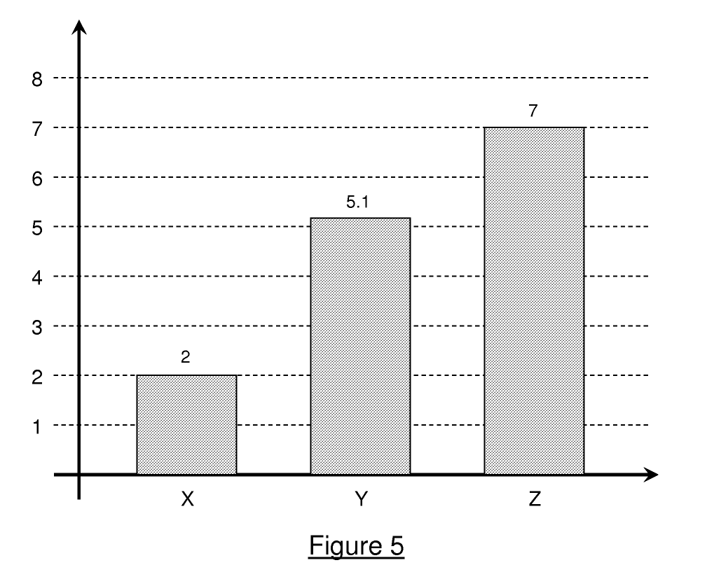

FIG. 5 is graph showing typical threshold values for use in apparatus according to the embodiments of the present invention.

According to a first embodiment of the invention as shown in FIG. 1, there is provided an oscillator 1 coupled to an “aerial”, that is, a transmitter probe 2 which is arranged to generate a frequency of approximately 5 MHz. An array of receiver probes 3, 4, 5 receive the signal transmitted from the transmitter probe 2, and each receiver probe 3, 4, 5 is connected to a respective rectifier 6, 7, 8, such as a diode bridge, from which a signal is transmitted to a multi-channel analogue-to-digital converter (ADC) input to a microprocessor 9. Similarly, a signal directly derived from the oscillator is provided to an ADC, in order that the received signals may be normalized with the amplitude of the transmitted signal.

A storage device 10 connected to the microprocessor contains reference data such as the conductance and capacitance of various skin types, such as dry skin and oily skin. The storage device 10 further comprises reference data for the various skin types under different conditions, for example with and without the presence of a coupling gel. In addition, the storage device 10 further comprises a table that correlates skin type and condition, with the dosage of intense-pulsed-light that is required to treat the skin type. This means that the microprocessor 9 is able to provide a dosage that will be tolerated by the skin under its actual conditions, while obtaining the end result desired.

Referring to FIG. 2, there is shown a front view of apparatus that contains a discharge tube (not shown) for generating intense-pulsed-light. The tube is protected by a glass block 11, which is within a substantially rectangular aperture 12 of a frame, with the probes 13a to 13d being arranged each with an insulating support and positioned just outside the corners of the aperture 12.

Exemplary control circuitry, which controls the firing of the tube (not shown) in accordance with a second embodiment of the invention, is shown in FIG. 3, and includes probes 13a to 13d corresponding to the reference numerals used in FIG. 2).

The control circuit 14 shown in FIG. 3 comprises a processing unit 15 which receives directly as input from a power rail 16, a continuous train of periodic pulses of duration T. This input is used to establish a time reference frame for each of the subsequent inputs to the processing unit 15 via circuit paths 17a to 17d. Each circuit path 17a to 17d comprises a respective comparator 18 which separately receives as input, a voltage from the power rail 16. Each of the comparators 18 is arranged to generate a respective output to the processing unit 15 when the voltage at the respective input reaches a predetermined threshold, for example about 3.8V.

The voltage input to each comparator 18 is governed by a pair of resistors 19 each typically being about 100 kΩ, arranged in a series configuration with the respective comparator 18. Each of the four probes 13a to 13d is separately connected to a respective circuit path 17a to 17d at a position intermediate the respective pairs of series resistors 19. The probes 13a to 13d in the embodiment of FIG. 3 may be arranged in a manner such as that described above with reference to FIG. 2, and are each arranged to transmit and receive an electrical signal.

The distal end of each probe 13a to 13d may be initially located in free space (in contact with air) and will thus generate a small capacitance. Accordingly, the application of a voltage pulse to the power rail 16 will cause the voltage input to the respective comparator 18 to increase to the threshold voltage over a time interval t₁. If the probes 13a to 13d are all in contact with skin, then this will act to increase the effective capacitance of the probes 13a to 13d thereby causing an increase in the interval t₁.

In use, a periodic train of voltage pulses, each of magnitude approximately 5V and duration up to 100 μs is continuously applied to the power rail 16. At a time corresponding to the leading edge of each voltage pulse on the power rail 16, the voltage input to each comparator 18 will begin to increase. The rate of increase of the voltage input to each comparator 18 will depend upon whether the respective probe 13a to 13d is in free space or coupled to skin. In the latter case, the rate of increase of the voltage input to each comparator 18 will further depend on the condition of the skin and on whether a coupling gel has been applied to the skin.

Probes 13a to 13d will, when coupled to the skin, cause the voltage input to the respective comparator 18 to increase more slowly than for those comparators for which the probes are arranged in free space. Moreover, it is found that the application of a coupling gel to the skin further reduces the rate of voltage rise input to the respective comparator 18.

The processing unit 15 only outputs a signal to cause the discharge lamp (not shown) to generate a pulse of intense light when it is determined that all four probes 13a to 13d are in contact with skin. Accordingly, the time for the comparators 18 to change output state is monitored and only if the time taken for each comparator 18 to change output state is above a threshold time, as determined from the leading edge of a selected voltage pulse on the power rail 16, will the processing unit 15 instruct the firing of the discharge lamp (not shown). In this respect, it is not necessary for each comparator 18 to change output state. If the output from each and every comparator 18 does not change state due to an excessively long rise time, then this is further taken to indicate that the respective probe(s) 13a to 13d is/are in contact with the skin.

The continuous application of voltage pulses to the power rail 16 thus provides for a continuous verification of whether the probes 13a to 13d are in contact with skin, and thus the circuit 14 will inhibit the firing of the discharge lamp (not shown) if at least one of the probes 13a to 13d breaks or loses contact with the skin.

Referring to FIG. 4, there is shown an exemplary flow chart, showing the sequence of operations or steps in the method of controlling the operation of an intense-pulsed-light source according to the first or second embodiment of the present invention.

Block A represents the first step, namely the application of gel to the skin. Block B represents the second step, namely the application of an array of probes 2 to or 13a to 13d, to the skin.

A pulsed electrical signal is sent through the probes 2 to 5, or 13a to 13d in step C and the time to reach the threshold voltage for each probe 2 to 5, or 13a to 13d is assessed in step D. The time is compared to a stored threshold value in step E; when the time is found to be greater than the stored threshold value in step F, the processing unit 10 or 15 outputs a control signal to permit a discharge lamp (not shown) to fire. Conversely, when the time is determined to be less that the stored threshold value in step F, the firing of the discharge lamp (not shown) is inhibited.

The stored threshold value chosen will depend to some extent on the frequency of the applied voltage pulse. Accordingly, it is preferably to normalise the time. Normalised time values of about 5.1 were obtained using apparatus according to the second embodiment of the invention (with the probes 13a to 13d being both transmitters and receivers) with oily skin; about 5.4 with degreased skin; about 6.3 for sweaty skin and over 7.0 for gel-coated skin—depending on the nature of the skin to which the gel was applied.

The stored time, below which firing is not permitted, may therefore be based on the above values, such as about 4. In that case, the firing will be permitted even when the probes 13a to 13d are not in contact with gel-coated skin. Alternatively, the stored output time below which firing is not permitted, may be set at about 7; in which case the firing will only be permitted when the probes 13a to 13d are in contact with gel-coated skin.

FIG. 5 provides a graphical comparison of the normalised time taken for the comparators to change state, when the probes 13a to 13d are arranged in free space—as indicated by column X, when the probes 13a to 13d are arranged in contact with skin—as indicated by column Y, and when the probes are arranged in contact with skin to which a gel has been applied—as indicated by column Z.

If the normalised time is less than a first threshold (column Y), this is taken to be indicative of the absence of skin, whereas if it exceeds a second threshold this is taken to be indicative of the presence of gel-coated skin (column Z). An intermediate value corresponds to a value measured for oily skin. If the detected value is below the first threshold, an alarm is triggered or a feedback signal is provided to prevent inadvertent operation of an intense-pulsed-light source.

From the foregoing therefore, it is evident that the apparatus and method of the present invention can prevent inadvertent operation of an intense-pulsed-light source when, for example one or more of the tips of the probes fail to contact skin, or for example when they contact another part of a body, such as an eye or mucous membrane.

1. Apparatus for detecting the presence of skin, which apparatus comprises
a) a set of probes each having tips arranged to simultaneously touch the skin and define a predetermined pattern on the skin, at least one of said probes being arranged to transmit a pulsed electrical signal and at least one of said probes being arranged to receive said transmitted electrical signal;
b) signal detector means for detecting the or each received electrical signal;
c) means for comparing a numerical value obtained from at least one detected signal from said signal detector means with at least one predetermined numerical value; and
d) means for providing an output when said value obtained from the detected signal differs from said predetermined numerical value by more than a predetermined amount.

2. Apparatus according to claim 1, wherein the predetermined pattern comprises a rectangle defined by the tips of said probes.

3. Apparatus according to claim 1, wherein the probes comprise elongate pins each having a diameter of 2 to 3 mm.

4. Apparatus according to any of claim 1, wherein at least some of said probes are arranged both to transmit and receive said electrical signal.

5. Apparatus according to claim 4, wherein all of the set of probes are arranged both to transmit and receive said electrical signal.

6. Apparatus according to any of claim 1, in which one of said probes is arranged to transmit said electrical signal and the remainder of said probes are arranged to receive said signal.

7. Apparatus according to claim 1, in combination with an intense pulsed light source.

8. Apparatus according to claim 7, wherein said output comprises a feedback control preventing operation of said light source.

9. Apparatus for detecting the presence of skin, which apparatus comprises
a) a set of probes, each probe of the set of probes having a tip, the tip of each of said probes being arranged to simultaneously touch the skin and define a predetermined pattern on the skin, at least one of said probes being arranged to both transmit a pulsed electrical signal and to receive said transmitted electrical signal;
b) a signal detector for detecting the or each received electrical signal;
c) a comparator for comparing a numerical value obtained from at least one detected signal from said signal detector with at least one predetermined numerical value; and
d) a processor for providing an output when said value obtained from the detected signal differs from said predetermined numerical value by more than a predetermined amount.

10. Apparatus according to claim 9, wherein the predetermined pattern comprises a rectangle defined by the tips.

11. Apparatus according to claim 9, wherein the probes comprise elongate pins each having a diameter of 2 to 3 mm.

12. Apparatus according to claim 9, wherein each one of the set of probes is arranged both to transmit and receive said electrical signal.

13. Apparatus according to claim 9, in combination with an intense pulsed light source.

14. Apparatus according to claim 5, wherein said output comprises a feedback control for preventing operation of said light source.

15. A method of controlling the operation of an intense pulsed light source, the method comprising
(a) providing a set of probes and at least one signal detector, each probe of the set of probes having a tip,
(b) applying the set of probes such that the tips thereof simultaneously touch a test surface with the tips defining a predetermined pattern on the test surface,
(c) transmitting a pulsed electrical signal from at least one of said probes;
(d) receiving said signal by said at least one of said probes,
(e) providing connection of each of said probes receiving said transmitted pulsed electrical signal to a respective individual signal detector of the at least one signal detector,
(f) comparing a value of said detected signals from the or each signal detector with at least one predetermined value; and
(g) providing an output when the compared value is outside a predetermined range, which output either prevents or permits said operation of said light source.

16. A method according to claim 15, wherein the test surface comprises skin.

17. A method according to claim 16, wherein the skin has gel applied thereto.

18. A method of treating skin with an intense pulsed light source, in which the intense pulsed light source is controlled by a method according to claim 15.
